(19) United States
(12) Patent Application Publication
(10) Pub. No.: US 2023/0193353 A1
(43) Pub. Date: Jun. 22, 2023

(54) METHODS AND COMPOSITIONS FOR HIGH-FIDELITY SEQUENCE ANALYSIS OF INDIVIDUAL LONG AND ULTRALONG NUCLEIC ACID MOLECULES

(71) Applicant: Northeastern University, Boston, MA (US)

(72) Inventors: Zoe Fleischmann, Flourtown, PA (US); Konstantin Khrapko, Newton, MA (US); Dori C. Woods, Londonderry, NH (US); Jonathan L. Tilly, Windham, NH (US)

(21) Appl. No.: 17/923,700

(22) PCT Filed: May 7, 2021
(86) PCT No.: PCT/US2021/031317

§ 371 Date: Nov. 7, 2022

Related U.S. Application Data
(60) Provisional application No. 63/021,173, filed on May 7, 2020.

(51) Int. Cl. C12Q 1/6806; C12Q 1/6876

(52) U.S. Cl. CPC C12Q 1/6806; C12Q 1/6876

(57) ABSTRACT

Disclosed are compositions and methods related to the use of plurality of reverse transcriptase primers, unique molecular identifiers (UMIs), and/or spiky primers with unique junction identifiers to improve the sequencing and amplifications methods. In some embodiments, the disclosed methods can identify sequencing errors and PCR-jumping errors.

### RELATED APPLICATION

This application claims the benefit of priority to U.S. Provisional Patent Application Ser. No. 63/021,173, filed May 7, 2020.

### GOVERNMENT SUPPORT

This invention was made with government support under Grant Numbers HD091439 and AG012279 awarded by the National Institutes for Health, and Grant Number 1750996 awarded by the National Science Foundation. The government has certain rights in the invention.

### BACKGROUND

Accurate analysis of the precise order of nucleotides in deoxyribonucleic acid (DNA) and ribonucleic acid (RNA) molecules is fundamentally important to understanding the biology and function of all living organisms, as well as of organisms that exist outside the conventional definition of “living”, such as viruses. Since 1965, when the complete sequence of a nucleic acid was first reported, technologies for DNA sequencing have undergone a number of dramatic improvements. At present, next generation sequencing (NGS)—such as that offered by the Illumina platform, and third-generation sequencing—such as the single-molecule real-time (SMRT) platform offered by Pacific Biosciences (PacBio) or the nanopore platform offered by Oxford Nanopore Technologies (ONT), enable high-throughput sequencing of DNA molecules. Even with these advances, however, two major limitations in conventional approaches to DNA sequencing exist: 1) an inability to perform high-fidelity sequencing of individual long molecules of DNA for combinations of single nucleotide variants (SNVs) on a given molecule (referred to as phase or linkage); and, 2) confounding data management and interpretation issues associated with nucleotide sequence errors introduced as artefacts by various sequencing technologies. If the starting material is RNA, a third major limitation exists: an inability to efficiently reverse transcribe long and ultralong RNA molecules into first-strand complementary DNA (cDNA) for downstream analyses. This latter problem is complicated further if the RNA is viral in origin (compared to typical messenger RNA or mRNA transcripts), since viral RNA genomes often carry secondary structures. Hence, there is an urgent need to find new strategies to sequence nucleic acids.

### SUMMARY

Disclosed are methods for generating a DNA/RNA duplex from a target RNA molecule comprising incubating a plurality of reverse transcriptase primers (RT primers) and the target RNA molecule under conditions such that the target RNA molecule is reverse transcribed generating a DNA/RNA duplex, wherein the plurality of RT primers are complementary to multiple annealing sites of the target RNA molecule such that each RT primer has an annealing site that is different than the annealing site of another RT primer in the plurality. Numerous embodiments are further provided that can be applied to any aspect of the present invention described herein. For example, in some embodiments, the sequence of the target RNA molecule between two adjacent annealing sites is 1,000 to 7,000 nucleotides long, preferably the sequence of the target RNA molecule between two adjacent annealing sites is about 1,000, 1,500, 2,000, 2,500, 3,000, 3,500, 4,000, 4,500, 5,000, 5,500, 6,000, 6,500, or 7,000 nucleotides long.

In some embodiments, the method further comprising incubating an additional RT primer, wherein the additional RT primer comprises in 5′ to 3′ order: (a) a first generic primer region having a nucleotide sequence that is not complementary to a sequence of the target RNA, (b) a first unique molecular identifier (UMI-A) region, and (c) a RT primer region that is complementary to the sequence located at the 3′ end region of the target RNA. In some embodiments, the target RNA molecule is reverse transcribed via a reverse transcriptase, preferably the reverse transcriptase is a processive reverse transcriptase. In some embodiments, the reverse transcriptase reverse transcribes the sequence of the target RNA molecule between two adjacent annealing sites thereby generating complementary DNA fragments annealed to the target RNA molecule. In some embodiments, the reverse transcriptase further reverse transcribes the adjacent annealing site thereby replacing the 5′ end of the adjacent fragment and creating excess single-stranded DNA. In some embodiments, the method further comprising trimming the excess single-stranded DNA via single-stranded DNA-specific exonuclease. In some embodiments, the single-stranded DNA-specific exonuclease is single-stranded DNA-specific 3′-5′/5′-3′ exonuclease VII (ExoVII). In some embodiments, the method further comprising ligating the DNA fragments via ligase.

In some embodiments, the RT primer comprises in 5′ to 3′ order: (a) a first specific primer region having a nucleotide sequence that is complementary to a first annealing site of the target nucleic acid molecule; (b) a first unique junction identifier comprising random nucleotides; and (c) a second specific primer region having a nucleotide sequence that is complementary to a second annealing site of the target nucleic acid molecule, wherein the second annealing site is adjacent to the first annealing site. In some embodiments, the RT primer further comprises a second unique junction identifier comprising a nucleic acid sequence complementary to the first unique junction identifier. In some embodiments, there are no nucleotides between the first annealing site and the second annealing site of the target nucleic acid molecule. In some embodiments, there are 1-100 nucleotides between the first annealing site and the second annealing site of the target nucleic acid molecule. In some embodiments, the RT primer is DNA or RNA. In some embodiments, the target nucleic acid molecule is DNA or RNA.

In one aspect, discloses here is a method of generating a double-stranded cDNA molecule comprising the steps of: (a) generating a DNA/RNA duplex according to the method disclosed herein; (b) treating the DNA/RNA duplex with RNase thereby removing the RNA; and (c) incubating an adapter primer comprising a region that is complementary to the sequence located at the 3′ end region of the DNA under conditions such that a complementary DNA strand is formed thereby generating a double-stranded cDNA molecule. In some embodiments, the RNase is RNase-H. In some embodiments, the adapter primer, further comprises on the 5′ end in 5′ to 3′ order: (a) a region complementary to a second generic primer having a nucleotide sequence that is not complementary to a sequence of the cDNA, and (b) a region complementary to a second unique molecular identifier (UMI-B).

In some embodiments, the complementary DNA strand is formed via a DNA polymerase. In some embodiments, the DNA polymerase is T4 DNA polymerase. In some embodiments, the target RNA molecule is less than 1-kb in length. In some embodiments, wherein the target RNA molecule is between 1-kb to 5-kb in length. In some embodiments, the target RNA molecule is 1-kb, 2-kb, 3-kb, 4-kb, or 5-kb in length. In some embodiments, the target RNA molecule is between 5-kb to 10-kb in length. In some embodiments, the target RNA molecule is 6-kb, 7-kb, 8-kb, 9-kb, or 10-kb in length. In some embodiments, the target RNA molecule is between 10-kb to 15-kb in length. In some embodiments, the target RNA molecule is 11-kb, 12-kb, 13-kb, 14-kb, or 15-kb in length. In some embodiments the target RNA molecule is between 15-kb to 30-kb in length. In some embodiments, the target RNA molecule is 18-kb, 20-kb, 22-kb, 24-kb, 26-kb, 28-kb, or 30-kb in length. In some embodiments, the target RNA molecule is greater than 30-kb in length, In some embodiments, the target RNA molecule is present in a homogeneous sample comprising the same RNA molecules. In some embodiments, the target RNA molecule is present in a heterogeneous sample comprising two or more different RNA molecules. In some embodiments, the target RNA molecule is from a virus, a bacterium, a yeast cell, a fungal cell, a plant cell, or an animal cell. In some embodiments, the target RNA molecule is from a plant cell infected with a virus. In some embodiments, the target RNA molecule is from an animal cell infected with a virus.

In another aspect, disclosed herein is a method of detecting and removing an artificially recombined DNA molecule (chimera) resulting from PCR-jumping comprising: (a) generating a double-stranded cDNA molecule according to the method disclosed herein; (b) amplifying the double-stranded cDNA molecule via a polymerase chain reaction using a first primer and a second primer that are complementary to the first generic primer region and the second generic primer region, respectively; (c) sequencing the amplified double-stranded cDNA molecule; (d) detecting the artificially recombined DNA molecule which does not have both UMI-A and UMI-B on the same double-stranded cDNA molecule; and (e) removing the artificially recombined DNA molecule in silico.

In another aspect, disclosed herein is a nucleic acid primer for sequencing a region of a target nucleic acid molecule comprising, in 5′ to 3′ order: (a) a first specific primer region having a nucleotide sequence that is complementary to a first annealing site of the target nucleic acid molecule; (b) a first unique junction identifier comprising random nucleotides; (c) a first universal primer region having a nucleotide sequence that is not complementary to a sequence of the target nucleic acid molecule; (d) a second universal primer region having a nucleotide sequence that is not complementary to a sequence of the target nucleic acid molecule; (e) a second unique junction identifier comprising a nucleic acid sequence complementary to the first unique junction identifier; and (f) a second specific primer region having a nucleotide sequence that is complementary to a second annealing site of the target nucleic acid molecule, wherein the second annealing site is adjacent to the first annealing site. In some embodiments, there are no nucleotides between the first annealing site and the second annealing site of the target nucleic acid molecule. In some embodiments, there are 1-100 nucleotides between the first annealing site and the second annealing site of the target nucleic acid molecule. In some embodiments, the nucleic acid primer is DNA or RNA. In some embodiments, the target nucleic acid molecule is DNA or RNA.

In another aspect, disclosed herein is a nucleic acid primer for sequencing a region of a target nucleic acid molecule comprising, in 5′ to 3′ order: (a) a first specific primer region having a nucleotide sequence that is complementary to a first annealing site of the target nucleic acid molecule; (b) a first unique junction identifier comprising random nucleotides; and (c) a second specific primer region having a nucleotide sequence that is complementary to a second annealing site of the target nucleic acid molecule, wherein the second annealing site is adjacent to the first annealing site. In some embodiments, the nucleic acid further comprises a second unique junction identifier comprising a nucleic acid sequence complementary to the first unique junction identifier. In some embodiments, there are no nucleotides between the first annealing site and the second annealing site of the target nucleic acid molecule. In some embodiments, there are 1-100 nucleotides between the first annealing site and the second annealing site of the target nucleic acid molecule. In some embodiments, the nucleic acid primer is DNA or RNA. In some embodiments, the target nucleic acid molecule is DNA or RNA.

In another aspect, disclosed herein is a method of generating a nucleic acid product comprising incubating the nucleic acid primer disclosed herein and a target nucleic acid molecule under conditions such that the nucleic acid product is formed. In some embodiments, the nucleic acid product is formed via a DNA polymerase. In some embodiments, the nucleic acid product is formed via a reverse transcriptase. In some embodiments, the method further comprising incubating an adapter primer having a nucleotide sequence that is complementary to an annealing site that is downstream of the first annealing site of the target nucleic acid molecule, thereby generating a nascent nucleic acid strand upstream of the nucleic acid primer and creating a nick between the 5′ end of the nucleic acid primer and the 3′ end of the nascent nucleic acid strand. In some embodiments, the method further comprising ligating the 5′ end of the nucleic acid primer and the 3′ end of the nascent nucleic acid strand via ligase. In some embodiments, the method further comprising incubating a plurality of the nucleic acid primer of any one of claims 32-46, wherein each nucleic acid primer has a first annealing site and a second annealing site that are different than the first annealing site and the second annealing site of another nucleic acid primer in the plurality. In some embodiments, the target nucleic acid molecule is less than 1-kb in length. In some embodiments, the target nucleic acid molecule is between 1-kb to 5-kb in length. In some embodiments, the target nucleic acid molecule is 1-kb, 2-kb, 3-kb, 4-kb, or 5-kb in length. In some embodiments, the target nucleic acid molecule is between 5-kb to 10-kb in length. In some embodiments, the target nucleic acid molecule is 6-kb, 7-kb, 8-kb, 9-kb, or 10-kb in length. In some embodiments, the target nucleic acid molecule is between 10-kb to 15-kb in length. In some embodiments, the target nucleic acid molecule is 11-kb, 12-kb, 13-kb, 14-kb, or 15-kb in length. In some embodiments, the target nucleic acid NA molecule is between 15-kb to 30-kb in length. In some embodiments, the target nucleic acid molecule is 18-kb, 20-kb, 22-kb, 24-kb, 26-kb, 28-kb, or 30-kb in length. In some embodiments, the target nucleic acid molecule is greater than 30-kb in length. In some embodiments, the target nucleic acid molecule is present in a homogenous sample comprising the same nucleic acid molecules. In some embodiments, the target nucleic acid molecule is present in a heterogeneous sample comprising two or more different nucleic acid molecules. In some embodiments, the target nucleic acid molecule is from a virus, a bacterium, a yeast cell, a fungal cell, a plant cell, or an animal cell. In some embodiments, the target nucleic acid molecule is from a plant cell infected with a virus. In some embodiments, the target nucleic acid molecule is from an animal cell infected with a virus. In some embodiments, the target nucleic acid is a single stranded nucleic acid. In some embodiments, the target nucleic acid is a double stranded nucleic acid. In some embodiments, the target nucleic acid is a linear nucleic acid. In some embodiments, the target nucleic acid is a circular nucleic acid.

In another aspect, disclosed herein is a method of identifying the sequence of a target nucleic acid comprising: (a) generating a nucleic acid product according to the method disclosed herein; (b) incubating a first specific primer and a second specific primer that are complementary to the first specific primer region and the second specific primer region of the nucleic acid primer and the nucleic acid product under conditions such that the nucleic acid product is amplified, thereby generating nucleic acid fragments that are flanked with unique junction identifiers; (c) sequencing the nucleic acid fragments; (d) assembling the nucleic acid fragments in silico, thereby identifying the sequence of the target nucleic acid.

### BRIEF DESCRIPTION OF THE DRAWINGS

FIG. 1 shows first-strand cDNA synthesis. Several RNA molecule-specific oligonucleotide primers (RT primers, highlighted in this figure in gray; this figure shows four) that are complementary to multiple regions distributed along the entire length of the RNA template of interest are used to independently prime multiple RT reactions on the same molecule. Once the RTase reaches the next RT primer annealing site, the enzyme will push away and replace the 5′ end of the previous reverse-transcript in front of it and continue to reverse transcribe the first-strand cDNA into the next region. Any excess of single-stranded cDNA is then trimmed using single-stranded DNA-specific exonuclease VII (ExoVII). Subsequent ligation of the resulting nicked RNA/DNA duplex using Taq ligase, followed by RNase-H treatment to remove the RNA template, produces a continuous first-strand cDNA covering the entire template sequence. One UMI which is 5′ with respect to the cDNA sequence (UMI-A) is attached to the cDNA via the first (most upstream) of the RT primers used to make the cDNA. It also attaches a 5′-generic primer (Generic Primer A). The 3′-terminal UMI ((UMI-B) is attached using a non-extendable sequence-specific adapter with a 3′-ddN nucleotide. A portion of this adapter that is complementary to the RNA sequence anneals to the 3′-end of the cDNA and allows extension of the 3-'end to include the second UMI (UMI-B) and a 3′-end generic primer (Generic Primer B).

FIG. 2 shows methods to detect and remove, in silico, chimeras that result from PCR-jumping. two original template molecules—molecule k and molecule n of many template molecules in the mixture are depicted. The cDNA population representing each molecule (i.e., k or n) consists of a “core” of non-jumped sequences that do not exhibit artificial recombination between molecules, all of which carry the original combination of UMIs (e.g. UMI-Aₖ and UMI-Bₖ for molecule k, and UMI-Aₙ and for UMI-Bₙ molecule n). However, PCR-jumping results in an admixture of non-original UMI combinations as well (e.g., UMI-Aₖ/UMI-Bₙ, representing a chimera formed through recombination between molecules k and n).

FIG. 3 shows spiky primers, which are defined as nucleic acid constructs comprising DNA and/or RNA, each of which consists of three main features: (1) Two anti-parallel, non-complementary oligonucleotide “feet,” (2) a double-stranded fully complementary region with random nucleotides that serve as a unique junction identifier (UJI), and its reverse complement, and (3) a region that incorporates universal primer sequences.

FIGS. 4A-4E show synthesis of spiky primers. FIG. 4A shows the pre-synthesized components of spiky primers: (1) oligo-1, with a UJI and 3′-foot; (2) oligo-2, with a 5′-foot and a sequence that is complementary to the sequence of oligo-1 between the UJI and the 3′-foot; and, (3) oligo-3, with a stem and a loop structure, that latter of which contains universal primer sequences. FIG. 4B shows that oligo-1 and oligo-2 are mixed and the complementary region on oligo-2 initiates formation of a double-stranded stem with oligo-1. Addition of polymerase extends the sequence from the 3′-end of oligo-2 to make a complete stem with a double-stranded UJI. The newly completed stem also contains a double-stranded restriction enzyme site. FIG. 4C shows restriction digestion to create sticky end. FIG. 4D shows ligation across free ends to incorporate linker. FIG. 4E shows completed spiky primers.

FIG. 5 shows annealing spiky primers (or alternative structures) to the DNA molecule of interest.

FIG. 6 shows spiky primer-based PCR (spiky-PCR) for elongation and ligation.

FIG. 7 shows removal of incomplete molecules and non-specific products.

FIG. 8 shows generic PCR of sub-fragments.

FIG. 9 shows analysis of circular nucleic acid molecules.

### DETAILED DESCRIPTION

Existing NGS platforms are designed for high-fidelity sequencing of short DNA fragments (<300-basepairs, bp). The “Deep Sequencing” or high-coverage version of Iliumina NGS can be used to explore microheterogeneity in DNA sequences, but this approach yields simply a list of nucleotide variants and their frequencies. It does not generate reliable information on linkage between variants (viz. which variants may be positioned on the same DNA molecule). Use of the Illumina “Phased Sequencing” platform, which employs a combination of long and short pair-ends, can be used to determine linkage of mutations in, for example, human genome sequencing analysis. However, Illumina Phased Sequencing requires large quantities of native DNA, and it cannot be used with applications that involve polymerase chain reaction (PCR) amplification of templates due to the issue of “PM-jumping”, or the formation of artifactual chimeras (recombinant molecules) resulting from artificial recombination between different DNA molecules. In contrast, the most advanced third-generation single-molecule sequencing technologies (e.g., ONT and PacBio) can produce much longer reads of DNA sequences, but inherent error rates in each approach are not compatible with high-fidelity analysis of SNVs in long DNA molecules. Additionally, both ONT and PacBio sequencing rely on very large consensus reads from multiple different molecules, generating nucleotide sequence information across genomes. However, heterogeneity across individual molecules is erased in the consensus sequence because low frequency mutations cannot be reliably differentiated from sequencing errors. The challenge of obtaining high fidelity, single-molecule sequence information across long spans of DNA is complicated even further when these analyses are performed with complex or heterogeneous nucleic acid mixtures.

In an attempt to reduce error rates associated with third-generation sequencing platforms that enable high throughput capacity and relatively long reads, the addition of ‘bell adapters’ to sequencing templates allows the templates to be read multiple times in a continuous circle, resulting in a highly accurate circular consensus sequence (CCS). Unfortunately, application of the CCS approach is limited to short DNA fragments due to constraints on how long the polymerase remains active. Likewise, further improvements in chemistry and software have enabled ONT sequencing platforms, such as MinION, to enable a per-read error rate less than 5%; however, this degree of artefactual error is still incompatible with performing high-fidelity reads of individual long DNA molecules that are required for many applications in healthcare and biotechnology. In parallel to these types of efforts to improve on DNA sequencing, unique molecular identifiers (UMIs)—random oligonucleotide sequences specific to individual molecules that were first introduced to count nucleic acid molecules in a sample, have been employed in error correction approaches for DNA sequencing. However, high-fidelity analysis of single nucleotide variant (SNV) combinations in individual long molecules, especially in individual molecules present in heterogeneous samples, remains a major challenge. Additionally, one of the most problematic technical issues with any long nucleic acid molecule analysis requiring use of the polymerase chain reaction (PCR)—the formation of chimeras due to artificial recombination between different DNA molecules through “PCR-jumping”, was not resolved by the use of a single UMI applied to the 5′-end for molecule labeling.

To overcome these current limitations in achieving highly accurate single long-molecule DNA sequencing, a new tool termed Long-molecule UMI-driven Consensus Sequencing (LUCS; US Patent Publication No. 20180371544) was developed. Equally compatible with either PacBio or ONT sequencing platforms, the LUCS technology utilizes a combination of 5′-UMIs and 3′-UMIs incorporated onto the respective ends of each individual molecule of DNA, permitting the construction of consensus genome sequences from analysis of individual long molecules irrespective of the complexity of the nucleic acid sample. Additionally, the use of paired UMIs—one on each end of the DNA molecule of interest, enables in-silico detection and removal of artificially recombined molecules (chimeras) resulting from PCR jumping, which as mentioned above is a widely known source of artefact or error associated with conventional sequence analysis of, in particular, long and ultralong molecules.

In recent studies, it has been demonstrated that the use of LUCS increases single-molecule sequencing accuracy of the ONT MinION platform from ˜85% to 99.99% (i.e., 10⁻⁴ errors/nucleotide). This vast improvement in accuracy over current DNA sequencing platforms is due in large part to an inherently high resistance of LUCS to errors introduced by use of PCR—such errors include artifactual nucleotide substitutions as well as formation of chimeras. Thus, LUCS represents a significant step in the evolution of single long-molecule nucleic acid sequence analysis. Specifically, in sequencing situations where PCR amplification is obligate (e.g., genomic analysis of single cells or a limited sample input, or of pathogens in clinical samples where the number of pathogen genomes is limiting), LUCS is superior for achieving the high-fidelity DNA sequence reads needed for these studies.

Despite the advantages offered by LUCS, high-accuracy coverage of ultralong individual DNA molecules (e.g., around 15-kb or greater) remains a significant challenge. This is especially true for efforts aimed at management of diseases, illnesses and health complications resulting from infections caused by RNA viruses with large genomes. Viruses such as these include SARS-CoV (Severe Acute Respiratory Syndrome Coronavirus), SARS-CoV-2 (Severe Acute Respiratory Syndrome Coronavirus-2 or COVID-19) and MERS (Middle East Respiratory Syndrome Coronavirus), as well as the “common cold” coronaviruses 229E, NL63, OC43 and HKU1, all of which possess genomes on the order of 30-kb. While the considerable size of a viral genome like this is highly problematic for detailed characterization studies, RNA viruses are especially challenging since the viral genome needs to be converted from an RNA format to a DNA format before downstream analyses can be conducted. The latter requires synthesis of a DNA strand that is complementary to the viral RNA genome template through reverse transcription (RT), using a reverse transcriptase (RTase) enzyme.

Importantly, the fidelity of the RT reaction for producing, without errors, a cDNA from an RNA genome of interest, and the subsequent nucleotide sequence analysis of that molecule, again without errors or artefacts, is crucial to defining genetic heterogeneity. Characterization of genetic variance across a population, whether it be a virus, a bacterium or a multi-cellular organism, is essential to understanding and predicting how populations react to stimuli. In particular, microbial and viral population dynamics remain poorly understood, despite advances in DNA sequencing technologies, because linkage information—or how one variant is related to another in the same molecule—is difficult to preserve across long molecules. Using current sequencing strategies, viral and bacterial populations are commonly represented by a single consensus genome sequence. This is a fundamentally flawed representation of populations that are far more dynamic and variable. Viral infections, for example, are initiated by viral “clouds”, not clonal expansion of a single viral particle. Viral populations exhibit population structures of co-existing quasispecies, which are minor subtypes characterized by multiple genetic variants, that co-exist in a single organism. Quasispecies are not typically evident when using consensus sequencing approaches that provide information on average genomes, not single molecules; however, accurate identification of quasispecies has major ramifications on the effectiveness of clinical interventions. For example, if only the major subtype(s) of a given virus is (are) targeted, non-targeted quasispecies will be able to evade surveillance and continue to circulate, eventually rendering expensive treatments obsolete and vaccines ineffective.

It is becoming increasingly clear that viral infections, for example, occur and progress as a result of dynamic viral clouds, not static genetic entities. As a consequence, treatment protocols for viruses such as human immunodeficiency virus (HIV) are multi-pronged, multi-drug cocktails. While this approach prolongs effectiveness of treatment, it is not a cure, and many viral particles are capable of evading the effects of the drugs. Similarly, vaccines are designed to target known viral epitopes. Any limitation in the scope of targeted epitopes, subtypes or quasispecies will severely limit the ability of a vaccine to contain or eliminate an outbreak. Furthermore, the population structure of actively developing outbreaks may help explain differences in patient presentation, and therefore inform the likelihood of success for different clinical treatments. The viral phylogeny—including characteristics such as phylogenetic diversity, number of subtypes, branch length and structure—is likely related to the trajectory of an infection. Put simply, an infection by a virus with limited genetic variance would be more easily cleared by a patient's innate immune system compared to an infection that exhibits early genetic diversity and rapid evolution. Resource allocation, such as the need for intensive care, might be more accurately modeled and predicted based on the degree of genetic diversity early in the infection, and treatments could begin earlier instead of waiting for symptoms to worsen.

The accurate study of RNA viruses is therefore crucial to successful management of viral infections through development of diagnostic tools to identify: those individuals who are infected, treatment strategies to fight the virus in infected individuals, and effective vaccines to prevent future infection, all of which depend on high-fidelity analysis of viral genomes (including characterization of natural recombination events that occur during viral evolution) and quasispecies (viral genetic variants) within infected individuals on a case-by-case basis (viz. intraindividual analysis). This, in turn, requires determination of nucleotide sequences of entire genomes of individual viral particles with extremely high fidelity. To this end, the group II intron maturase RTase from Eubacterium rectale, referred to as MarathonRT, is a highly-processive RTase which efficiently copies RNA transcripts. Although its processivity is superior to commercial RTases, such as Superscript IV, published studies of MarathonRT for use in analysis of human immunodeficiency virus (HIV) have shown the extent of its coverage in actual practice is around 10-kb. While the efficiency of MarathonRT to accurately and completely transcribe very long RNA templates is still not unequivocally established, empirical testing data available thus far for this high-processivity polymerase indicates that it will not be useful for transcribing ultralong RNA templates of coronaviruses, which approach 30-kb in length. Even if this is somehow achieved, no existing platform exists that would enable subsequent high-fidelity sequence analyses of individual ultralong cDNA molecules prepared from such RNA templates. Hence, there is an urgent need to find new strategies to sequence nucleic acids.

Disclosed are compositions and methods related to the use of plurality of reverse transcriptase primers, unique molecular identifiers (UMIs), and/or spiky primers with unique junction identifiers to improve the sequencing and amplifications methods. In some embodiments, the disclosed methods can identify sequencing errors and PCR-jumping errors.

In one embodiment, the invention can be used for the synthesis of continuous cDNA molecules from individual long and ultralong RNA molecules through “piecewise reverse transcription” (referred to hereafter as pRT). This technological advance over all existing methods of RT utilizes oligonucleotide primers complementary to, and spaced along, an entire RNA template irrespective of its overall length, which then facilitate multiple and independent, but partial-coverage, RI reactions in parallel. This produces a group of first-strand cDNA molecules spanning all areas of the RNA target that are then trimmed and ligated in sequence to generate a single first-strand cDNA covering the entire length of a desired RNA template with high efficiency and accuracy.

Methods of the invention can therefore, among other things, bypass enzymatic processivity limitations of all currently known RTases to efficiently cover and reverse transcribe long and ultralong RNA molecules.

In another embodiment, the invention enables high-fidelity sequence analysis of individual long and ultralong DNA molecules through “spiky-PCR”. This technological advance over all existing methods of single long-molecule nucleic acid analysis breaks apart or fragments a long or ultralong DNA molecule of interest into a series of sub-fragments, each of which can then be amplified by PCR with very high efficiency. Specifically, by inserting unique junction identifier (UJI) sequences that demarcate prospective DNA sub-fragments, an individual DNA molecule can be broken into UJI-labeled sub-fragments for high fidelity PCR and sequencing. Once the nucleotide sequence of each sub-fragment is obtained, the sequence of an individual long or ultralong DNA molecule can be reconstructed in full, such that the linkage between two or more nucleotide variants within the original DNA molecule of interest can be determined without limitations on the length of the original molecule. Fragmentation of long or ultralong DNA molecules into segments labeled with UJIs prior to PCR, and then aligning the amplified fragments for sequence reconstruction through their respective UJIs, therefore enables high-fidelity sequencing of long and ultralong DNA molecules.

In yet another embodiment of the invention, spiky-PCR can be used to detect or reject the presence of recombinants in a sample containing long divergent genomes, such as DNA in a microbiome sample or DNA in a population of viruses with quasispecies in a clinical sample. By subjecting the sample containing the mixture of genomes to spiky-PCR, the sequence of each individual molecule is then recovered for in silico analysis of the absence or presence of recombinant molecules. If desired, all artificial recombinants (i.e., those arising as a technology artefact) can be identified and removed in silico, with linkage of remaining molecules preserved. The ability of spiky-PCR to definitively identify and eliminate artificial recombinants (chimeras) from further analysis therefore enables identification and high-fidelity characterization of any natural or in-vivo recombination that has occurred in a population of molecules under study.

Methods of the invention can also be used to distinguish individual long-molecule sequences within mixed or heterogeneous nucleic acid pools, and subsequently enable high-fidelity sequence analysis of these individual molecules.

Methods of the invention are particularly applicable to, for example, the study of viral genomes, long and ultralong RNA molecules, microbial communities, mitochondrial genomes (i.e., mitochondrial DNA or mtDNA), and nuclear genomes (i.e., nuclear DNA).

In addition, methods of the invention can be used to perform high-accuracy genetic heterogeneity studies of associated SNVs in individual nucleic acid molecules of bacterial or viral sources, many of which have genomes that are typically longer than 10-kb.

In yet another embodiment, the invention can be used for the identification of SNV combinations in individual viral genomes at very low frequencies (e.g., even only a few SNVs per 10-kb or so of a molecule), as well as for detailed characterization of microheterogeneity in viral quasispecies, the latter of which is highly relevant to understanding, and effectively managing, fast-moving viral disease outbreaks, pandemics and endemics.

Methods of the invention can also be used to, for example, characterize microbiomes in individual organisms and in the environment, detect and analyze mtDNA heteroplasmy, and provide detailed genetic information in samples where nuclear DNA is unstable, such as in cells that are transforming into, or have acquired, a hyperplastic or cancerous state.

In a different embodiment of the invention, linked mutations (i.e., mutations occurring within a single molecule) can be identified with high accuracy, which is critical to many biological realms, including, but not limited to understanding epistatic interactions in disease, lineage tracing and phylogenetic analysis, and characterization of heterogeneity in mixed nucleic acid populations with unique genetic information.

Additionally, methods of the invention enable sequence analysis of continuous segments of RNA or DNA molecules, the latter of which in either a linear or a circular configuration, without being bound by processivity limitations of polymerases used for PCR amplification.

In yet another embodiment, methods of the invention can be used to identify and characterize nucleic acid recombination events (recombinant molecules or chimeras), whether occurring naturally in organisms through development and evolution or as an artefact of PCR-jumping associated with conventional nucleic acid amplification and sequencing technologies.

In a different embodiment, the invention enables definitive identification of nucleic acid subgroups in a sample based on their linked mutations and all associated diversity; the latter can be SNVs that are linked to some, but not all, of a given combination of linked variants. Through this embodiment, methods of the invention therefore enable analysis of, for example, differential evolutionary rate and selection pressure between different quasispecies, microheterogeneity in bacterial subgroups (e.g., cultured colonies, samples with many distinct subtypes), and microheterogeneity of populations with complex population structures (e.g., genetic selection in plants to optimize viability of germ cells).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

As used herein, two nucleic acid sequences “complement” one another or are “complementary” to one another if they base pair one another at each position.

As used herein, two nucleic acid sequences “correspond” to one another if they are both complementary to the same nucleic acid sequence.

As used herein, the Tm or melting temperature of two oligonucleotides is the temperature at which 50% of the oligonucleotide/targets are bound and 50% of the oligonucleotide target molecules are not bound. Tm values of two oligonucleotides are oligonucleotide concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

Processive reverse transcriptase, the processivity of a reverse transcriptase refers to the number of nucleotides incorporated in a single binding event of the enzyme. Therefore, a highly processive reverse transcriptase can synthesize longer cDNA strands in a shorter reaction time. Some reverse transcriptases can add as many as 1,500 nucleotides in a single binding event.

The term “in silico” is used to mean experimentation performed by computer.

The main difference between upstream and downstream DNA is that the upstream DNA is the DNA, which occurs towards the 5′ end from a particular point on the DNA strand whereas the downstream DNA is the DNA, which occurs towards the 3′ end.

A “nick” is a discontinuity in a double stranded DNA molecule where there is no phosphodiester bond between adjacent nucleotides of one strand typically through damage or enzyme action.

### Additional Exemplary Embodiments

In exemplary embodiment 1, provided herein is a method for the synthesis of a continuous cDNA strand from an individual RNA molecule through piecewise reverse transcription (pRT).

In exemplary embodiment 2, provided herein is the method of embodiment 1, wherein the length of the RNA molecule is less than 1-kb in length, is between 1-kb to 5-kb in length, is between 5-kb to 10-kb in length, is between 10-kb to 15-kb in length, is between 15-kb to 30-kb in length, or is greater than 30-kb in length.

In exemplary embodiment 3, provided herein is the method of embodiment 1, wherein the RNA molecule is present in a homogenous sample comprising the same RNA molecules. Or is present in a heterogeneous sample comprising two or more different RNA molecules.

In exemplary embodiment 4, provided herein is the method of embodiment 1, wherein the RNA molecule is from a virus, is from a bacterium, is from a yeast cell, is from a fungal cell, is from a plant cell, is from an animal cell, is from a plant cell infected with a virus, is from an animal cell infected with a virus, is produced inside a virus, bacterium, yeast cell, fungal cell, plant cell or animal cell in vivo, is produced inside a virus, bacterium, yeast cell, fungal cell, plant cell or animal cell in vitro, is produced outside a virus, bacterium, yeast cell, fungal cell, plant cell or animal cell, is produced inside an artificially engineered cell or vesicle, is produced outside a virus, bacterium, yeast cell, fungal cell, plant cell or animal cell, or is produced outside an artificially engineered cell or vesicle.

In exemplary embodiment 5, provided herein is the method of embodiment 4, wherein the animal cell is from any non-human animal species including, but not limited to, any species of insects, reptiles, amphibians, fish, birds, and non-human mammals.

In exemplary embodiment 6, provided herein is the method of embodiment 4, the animal cell is a human cell.

In exemplary embodiment 7, provided herein is the method of embodiment 6, wherein the animal cell infected with a virus is from any non-human animal species including, but not limited to, any species of insects, reptiles, amphibians, fish, birds, and non-human mammals

In exemplary embodiment 8, provided herein is the method of embodiment 7, wherein the animal cell infected with a virus is a human cell.

In exemplary embodiment 9, provided herein is a method for high-accuracy nucleotide sequencing of a nucleic acid molecule by spiky-PCR.

In exemplary embodiment 10, provided herein is the method of embodiment 9, wherein the method is used to sequence a single DNA molecule, or is used to sequence two or more DNA molecules.

In exemplary embodiment 11, provided herein is the method of embodiment 9, wherein the method employs unique junction identifier (UJI) nucleotide sequences, or employs spiky-PCR primers, each of which comprises template primer regions, UJI regions and universal primer regions.

In exemplary embodiment 12, provided herein is the method of embodiment 9, wherein the spiky PCR primers are used with a DNA molecule, or are used with an RNA molecule to generate a DNA molecule complementary to the target RNA molecule.

In exemplary embodiment 13, provided herein is the method of embodiment 12, wherein the RNA molecule is a viral RNA molecule, is a messenger RNA (mRNA) molecule, or is a long non-coding RNA (LncRNA) molecule.

In exemplary embodiment 14, provided herein is the method of embodiment 12, wherein the length of the RNA molecule is less than 1-kb in length, is between 1-kb to 5-kb in length, is between 5-kb to 10-kb in length, is between 10-kb to 15-kb in length, is between 15-kb to 30-kb in length, or is greater than 30-kb in length.

In exemplary embodiment 15, provided herein is the method of embodiment 12, wherein the RNA molecule is present in a homogenous sample comprising the same RNA molecules, or is present in a heterogeneous sample comprising two or more different RNA molecules.

In exemplary embodiment 16, provided herein is the method of embodiment 12, wherein the RNA molecule is from a virus, is from a bacterium, is from a yeast cell, is from a fungal cell, is from a plant cell, is from an animal cell, is from a plant cell infected with a virus, is from an animal cell infected with a virus, or is produced inside a virus, bacterium, yeast cell, fungal cell, plant cell or animal cell in vivo.

In exemplary embodiment 17, provided herein is the method of embodiment 12, wherein the RNA molecule is produced inside a virus, bacterium, yeast cell, fungal cell, plant cell or animal cell in vitro, is produced outside a virus, bacterium, yeast cell, fungal cell, plant cell or animal cell, is produced inside an artificially engineered cell or vesicle, molecule is produced outside a virus, bacterium, yeast cell, fungal cell, plant cell or animal cell, or is produced outside an artificially engineered cell or vesicle.

In exemplary embodiment 18, provided herein is the method of embodiment 17, wherein the animal cell is from any non-human animal species including, but not limited to, any species of insects, reptiles, amphibians, fish, birds, and non-human mammals.

In exemplary embodiment 19, provided herein is the method of embodiment 18, wherein the animal cell is a human cell.

In exemplary embodiment 20, provided herein is the method of embodiment 19, wherein the animal cell infected with a virus is from any non-human animal species including, but not limited to, any species of insects, reptiles, amphibians, fish, birds, and non-human mammals, or the animal cell infected with a virus is a human cell.

In exemplary embodiment 21, provided herein is the method of any preceding embodiments, wherein annealing of primers to a target DNA molecule is at a predetermined site or at predetermined sites on the target DNA molecule based on a priori knowledge of the target DNA molecule nucleotide sequence, the annealing of primers to a target DNA molecule is at an unknown site or at unknown sites on the target DNA molecule through the use of random oligonucleotide primer sequences, annealing of primers to a target RNA molecule is at a predetermined site or at predetermined sites on the target RNA molecule based on a priori knowledge of the target RNA molecule nucleotide sequence, or the annealing of primers to a target RNA molecule is at an unknown site or at unknown sites on the target RNA molecule through the use of random oligonucleotide primer sequences.

In exemplary embodiment 22, provided herein is the method of any preceding embodiments, wherein the length of the DNA molecule is less than 1-kb in length, is between 1-kb to 5-kb in length, is between 5-kb to 10-kb in length, is between 10-kb to 15-kb in length, is between 15-kb to 30-kb in length, or is greater than 30-kb in length.

In exemplary embodiment 23, provided herein is the method of any preceding embodiments, wherein the DNA molecule is linear, or molecule is circular.

In exemplary embodiment 24, provided herein is the method of any preceding embodiments, wherein the DNA molecule is present in a homogenous sample comprising the same DNA molecules, or is present in a heterogeneous sample comprising two or more different DNA molecules.

In exemplary embodiment 25, provided herein is the method of any preceding embodiments, wherein the method is not limited by processivity of DNA polymerases.

In exemplary embodiment 26, provided herein is the method of any preceding embodiments, wherein the DNA molecule is a single-stranded DNA molecule, is a double-stranded DNA molecule, is a nuclear DNA molecule, is a mitochondrial DNA molecule, or is a complementary DNA molecule.

In exemplary embodiment 27, provided herein is the method of any preceding embodiments, wherein the DNA molecule is from a virus, is from a bacterium, is from a yeast cell, is from a fungal cell, is from a plant cell, is from an animal cell, is from a plant cell infected with a virus, or is from an animal cell infected with a virus.

In exemplary embodiment 28, provided herein is the method of any preceding embodiments, wherein the DNA molecule is produced inside a virus, bacterium, yeast cell, fungal cell, plant cell or animal cell in vivo, is produced inside a virus, bacterium, yeast cell, fungal cell, plant cell or animal cell in vitro, is produced outside a virus, bacterium, yeast cell, fungal cell, plant cell or animal cell, is produced inside an artificially engineered cell or vesicle, is produced outside a virus, bacterium, yeast cell, fungal cell, plant cell or animal cell, or is produced outside an artificially engineered cell or vesicle.

In exemplary embodiment 29, provided herein is the method of any preceding embodiments, wherein the method is used to produce nucleotide sequence information for a DNA molecule, is used to produce nucleotide sequence information for an RNA molecule, is used to produce nucleotide sequence information for a viral RNA molecule, is used to produce nucleotide sequence information for a messenger RNA (mRNA) molecule, or is used to produce nucleotide sequence information for a long non-coding RNA (LncRNA) molecule.

In exemplary embodiment 30, provided herein is the method of any preceding embodiments, the animal cell is from any non-human animal species including, but not limited to, any species of insects, reptiles, amphibians, fish, birds, and non-human mammals.

In exemplary embodiment 31, provided herein is the method of any preceding embodiments, wherein the animal cell is a human cell.

In exemplary embodiment 32, provided herein is the method of any preceding embodiments, wherein the animal cell infected with a virus is from any non-human animal species, including, but not limited to, any species of insects, reptiles, amphibians, fish, birds, and non-human mammals.

In exemplary embodiment 33, provided herein is the method of any preceding embodiments, wherein the animal cell infected with a virus is a human cell.

In exemplary embodiment 34, provided herein is the method of any preceding embodiments, wherein the method is used for high throughput sequencing of pooled DNA molecules, is used for amplification of a single DNA molecule from any source with a known consensus sequence, or is used for single-molecule PCR when a target DNA molecule is not contiguous.

In exemplary embodiment 35, provided herein is a method to definitively detect or reject the presence of a recombinant DNA molecule in a sample containing divergent genomes.

In exemplary embodiment 36, provided herein is the method of any preceding embodiments, wherein an artificial recombinant DNA molecule—representing an artefact of the use of a technology, versus a natural recombinant DNA molecule—representing a nucleic acid produced as a result of biological processes occurring within living and non-living organisms, in a sample can be definitively distinguished and segregated from each other for separate analysis.

In exemplary embodiment 37, provided herein is the method of any preceding embodiments, wherein the length of the recombinant DNA molecule is less than 1-kb in length, is between 1-kb to 5-kb in length, is between 5-kb to 10-kb in length, is between 10-kb to 15-kb in length, is between 15-kb to 30-kb in length, or is greater than 30-kb in length.

In exemplary embodiment 38, provided herein is the method of any preceding embodiments, wherein the recombinant DNA molecule is a single-stranded DNA molecule, or is a double-stranded DNA molecule.

In exemplary embodiment 39, provided herein is the method of any preceding embodiments, wherein the recombinant DNA molecule is a nuclear DNA molecule, molecule is a mitochondrial DNA molecule, is a complementary DNA molecule, or is a complementary DNA molecule reversed transcribed from an RNA molecule.

In exemplary embodiment 40, provided herein is the method of any preceding embodiments, wherein the recombinant DNA molecule is from a virus.

In exemplary embodiment 41, provided herein is the method of any preceding embodiments, wherein the recombinant DNA molecule is a complementary DNA molecule reversed transcribed from an RNA molecule.

In exemplary embodiment 42, provided herein is the method of any preceding embodiments, wherein the recombinant DNA molecule is from a bacterium, is from a yeast cell, is from a fungal cell, is from a plant cell, is from an animal cell, is from a plant cell infected with a virus, or is from an animal cell infected with a virus.

In exemplary embodiment 43, provided herein is the method of any preceding embodiments, wherein the recombinant DNA molecule is produced inside a virus, bacterium, yeast cell, fungal cell, plant cell or animal cell in vivo, is produced inside a virus, bacterium, yeast cell, fungal cell, plant cell or animal cell in vitro, is produced outside a virus, bacterium, yeast cell, fungal cell, plant cell or animal cell, is produced inside an artificially engineered cell or vesicle, molecule is produced outside a virus, bacterium, yeast cell, fungal cell, plant cell or animal cell, or is produced outside an artificially engineered cell or vesicle.

In exemplary embodiment 44, provided herein is the method of any preceding embodiments, wherein the method is used to produce nucleotide sequence information for a recombinant DNA molecule, is used to produce nucleotide sequence information for an RNA molecule, is used to produce nucleotide sequence information for a viral RNA molecule, method is used to produce nucleotide sequence information for a messenger RNA (mRNA) molecule, or is used to produce nucleotide sequence information for a long non-coding RNA (LncRNA) molecule.

In exemplary embodiment 45, provided herein is the method of any preceding embodiments, wherein the animal cell is from any non-human animal species including, but not limited to, any species of insects, reptiles, amphibians, fish, birds, and non-human mammals.

In exemplary embodiment 46, provided herein is the method of any preceding embodiments, wherein the animal cell is a human cell.

In exemplary embodiment 47, provided herein is the method of any preceding embodiments, wherein the animal cell infected with a virus is from any non-human animal species, including, but not limited to, any species of insects, reptiles, amphibians, fish, birds, and non-human mammals.

In exemplary embodiment 48, provided herein is the method of any preceding embodiments, wherein the animal cell infected with a virus is a human cell.

In exemplary embodiment 49, provided herein is a method for the detection and in-silico removal of an artificially recombined DNA molecule (chimera) resulting from PCR-jumping during analysis of long and ultralong nucleic acid molecules in a sample.

In exemplary embodiment 50, provided herein is the method of any preceding embodiments, wherein the long or ultralong nucleic acid molecule is between 5-10-kb in length, is between 10-15-kb in length, is between 15-30-kb in length, or is greater than 30-kb in length.

In exemplary embodiment 51, provided herein is a method for the identification of single nucleotide variant (SNV) combinations in an individual nucleic acid molecule occurring at very low frequencies.

In exemplary embodiment 52, provided herein is the method of any preceding embodiments, wherein the frequency is between 100-500 SNVs per 10-kb of a single molecule, is between 50-100 SNVs per 10-kb of a single molecule, is between 10-50 SNVs per 10-kb of a single molecule, or is between 1-10 SNVs per 10-kb of a single molecule.

In exemplary embodiment 53, provided herein is a method for the identification of linked nucleotide mutations occurring within a single nucleic acid molecule.

In exemplary embodiment 54, provided herein is the method of any preceding embodiments, wherein the method enables definitive identification of nucleic acid subgroups in a sample based on their linked mutations.

In exemplary embodiment 55, provided herein is the method of any preceding embodiments, wherein the length of the nucleic acid molecule is less than 1-kb in length, is between 1-kb to 5-kb in length, is between 5-kb to 10-kb in length, is between 10-kb to 15-kb in length, is between 15-kb to 30-kb in length, or is greater than 30-kb in length.

In exemplary embodiment 56, provided herein is the method of any preceding embodiments, wherein the nucleic acid molecule is a single-stranded DNA molecule, is a double-stranded DNA molecule, is a nuclear DNA molecule, is a mitochondrial DNA molecule, or is a complementary DNA molecule.

In exemplary embodiment 57, provided herein is the method of any preceding embodiments, wherein the complementary DNA molecule is reversed transcribed from an RNA molecule.

In exemplary embodiment 58, provided herein is the method of any preceding embodiments, wherein the RNA molecule is from a virus.

In exemplary embodiment 59, provided herein is the method of any preceding embodiments, wherein the nucleic acid molecule is from a virus, is from a bacterium, is from a yeast cell, is from a fungal cell, is from a plant cell, is from an animal cell, is from a plant cell infected with a virus, is from an animal cell infected with a virus, is produced inside a virus, bacterium, yeast cell, fungal cell, plant cell or animal cell in vivo, is produced inside a virus, bacterium, yeast cell, fungal cell, plant cell or animal cell in vitro, molecule is produced outside a virus, bacterium, yeast cell, fungal cell, plant cell or animal cell, is produced inside an artificially engineered cell or vesicle, is produced outside a virus, bacterium, yeast cell, fungal cell, plant cell or animal cell, or is produced outside an artificially engineered cell or vesicle.

In exemplary embodiment 60, provided herein is the method of any preceding embodiments, wherein the method is used to produce nucleotide sequence information for a nucleic acid molecule.

In exemplary embodiment 61, provided herein is the method of any preceding embodiments, wherein the nucleic acid molecule is an RNA molecule, is a viral RNA molecule, is a messenger RNA (mRNA) molecule, or is a long non-coding RNA (LncRNA) molecule.

In exemplary embodiment 62, provided herein is the method of any preceding embodiments, the animal cell is from any non-human animal species including, but not limited to, any species of insects, reptiles, amphibians, fish, birds, and non-human mammals.

In exemplary embodiment 63, provided herein is the method of any preceding embodiments, wherein the animal cell is a human cell.

In exemplary embodiment 64, provided herein is the method of any preceding embodiments, wherein the animal cell infected with a virus is from any non-human animal species, including, but not limited to, any species of insects, reptiles, amphibians, fish, birds, and non-human mammals.

In exemplary embodiment 65, provided herein is the method of any preceding embodiments, wherein the animal cell infected with a virus is a human cell.

### EXAMPLES

The invention now being generally described, it will be more readily understood by reference to the following examples that are included merely for purposes of illustration of certain aspects and embodiments of the present invention, and are not intended to limit the invention.

### Example 1. Use of pRT to Synthesize a Continuous and Complete cDNA Molecule From a Template RNA Molecule

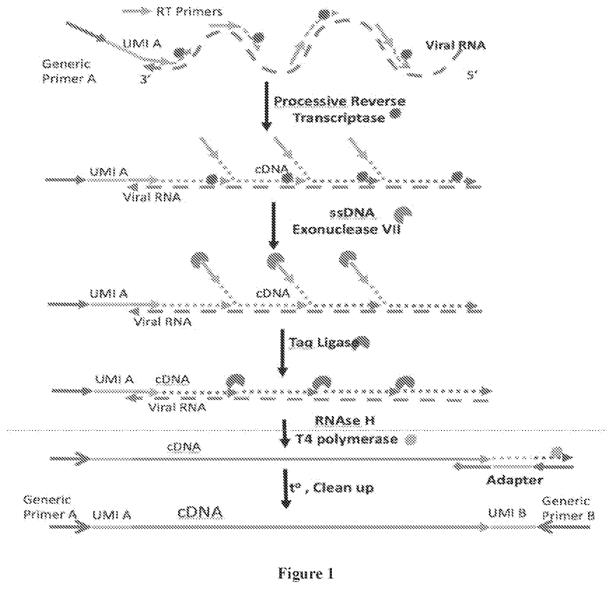

See FIG. 1. In this example of first-strand cDNA synthesis, several RNA molecule-specific oligonucleotide primers (RT primers, highlighted in this figure in gray for ease of visualization; this example shows four, but the actual number is determined by the length of the RNA molecule to be reverse-transcribed into cDNA) that are complementary to multiple regions distributed along the entire length of the RNA template of interest are used to independently prime multiple RT reactions on the same molecule, such that each region covered by a given RT reaction is no longer than 3-5-kb before it reaches the next downstream RT primer region. Once the RTase (in this example, MarathonRT is depicted) reaches the next RT primer annealing site, the enzyme will push away and replace the 5′ end of the previous reverse-transcript in front of it and continue to reverse transcribe the first-strand cDNA into the next region. The RTase will eventually stop and fall off the template; however, for pRT, it does not matter where this occurs once the next primed region ahead has been reached. Any excess of single-stranded cDNA is then trimmed using single-stranded DNA-specific 3′-5′/5′-3′ exonuclease VII (ExoVII). Subsequent ligation of the resulting nicked RNA/DNA duplex using Taq ligase, followed by RNase-H treatment to remove the RNA template, produces a continuous first-strand cDNA covering the entire template sequence. Note that one UMI which is 5′ with respect to the cDNA sequence (UMI-A) has already been attached to the cDNA at the previous stage via the first (most upstream) of the RT primers used to make the cDNA. This oligonucleotide primer is a UMI-bearing adapter, which attaches a molecule-specific tag to each cDNA molecule produced in this procedure. It also attaches a 5′-generic primer (Generic Primer A), which is used in subsequent PCR to preserve the attached UMI-A. The adapter portion of the first RT primer needs to be protected from ExoVII action with a complementary synthetic oligonucleotide (not shown in the example here). Alternatively, in other embodiments of the invention, attachment of a 5′-UMI (UMI-A) and 5′-generic primer (Generic Primer A) can be done via synthesis of an entire second cDNA strand from a gene-specific primer-adapter oligonucleotide using a highly-processive polymerase (e.g., Bacillus subtilis phage phi29, ϕ29).

The 3′-terminal UMI ((UMI-B) is attached using a non-extendable sequence-specific adapter with a 3′-ddN nucleotide to prevent elongation by the polymerase and creation of a complementary strand. A portion of this adapter that is complementary to the RNA sequence anneals to the 3′-end of the cDNA and allows extension of the 3-'end to include the second UMI (UMI-B) and a 3′-end generic primer (Generic Primer B). The adapter contains sequences complementary to UMI-B and to Generic Primer B, which are filled in by T4 polymerase to produce a complete cDNA flanked with molecule-specific UMIs and generic primers on both the 5′-end and the 3′-end of the molecule.

### Example 2. Identification and Resolution of PCR-Jumping Errors During Long and Ultralong Molecule Analysis to Ensure Molecular Continuity

Synthesis of individual molecules of cDNA from long or ultralong RNA templates does not serve its purpose unless the nucleotide sequence integrity of such molecules is maintained. Long and ultralong molecules are especially prone to artificial recombination, which generates chimeric cDNA molecules as an artefact. This process, often referred to as PCR-jumping, occurs when an incompletely amplified DNA fragment “primes” another fragment of DNA in a subsequent PCR cycle.

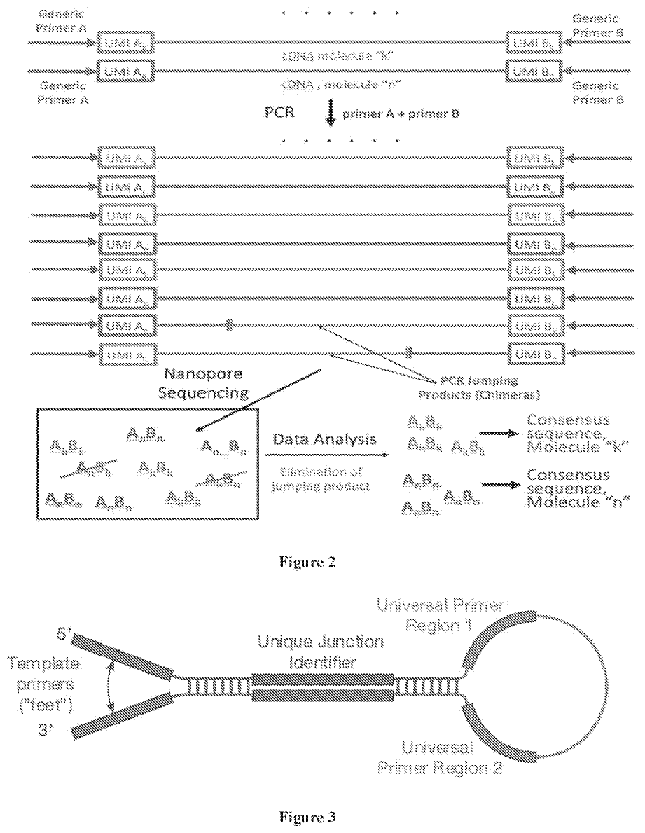

In the example of FIG. 2, methods to detect and remove, in silico, chimeras that result from PCR-jumping are illustrated through the use of UMIs attached to both ends (5′ and 3′) of the molecule of interest. Every original cDNA template subjected to PCR first establishes a clone of its daughter molecules in the absence of PCR-jumping. However, the likelihood, and frequency, of PCR-jumping increases as PCR cycle number increases. During later-stage PCR cycles, the concentration of PCR-generated cDNA strands, including a growing abundance of incomplete strands, increases, which then increases competition against oligonucleotide primers in priming nascent cDNA strands. When PCR-jumping starts to increase at later cycles, the clones are already large enough to enable one to distinguish “noise” due to PCR-jumping (viz. chimeric sequences) from the sequence of the original molecule.

For illustration, the FIG. 2 depicts two original template molecules—molecule k and molecule n of many template molecules in the mixture. At later-stage PCR cycles, a small but growing proportion of molecules have undergone artificial recombination due to PCR-jumping, leading to the generation of chimeras containing sequences from different molecules. At the end of the PCR process, the cDNA population representing each molecule (i.e., k or n) consists of a “core” of non-jumped sequences that do not exhibit artificial recombination between molecules, all of which carry the original combination of UMIs (e.g., UMI-Aₖ and UMI-Bₖ for molecule k, and UMI-Aₙ and UMI-Bₙ for molecule n). However, PCR-jumping results in an admixture of non-original UMI combinations as well (e.g., UMI-Aₖ/UMI-Bₙ, representing a chimera formed through recombination between molecules k and n). Because there are many different clones in the reaction, each particular non-original combination of UMIs (e.g., UMI-Aₖ/UMI-Bₙ) will be individually rare, allowing these artificial fragments to be easily distinguished and removed in silico from the original (“core”) sequences, the latter of which are present in high numbers. The utility of this dual-UMI-based technology, and its analytical pipeline, for detection and in-silico removal of chimeras during, for example, single-molecule mtDNA sequence analysis using LUCS has recently been demonstrated in practice by Annis et al. (Annis S., et al. Aging. 2020 Apr. 28; 12(8):7603-7613).

### Example 3. Description of Primers Used For Insertion of Unique Junction Identifiers (UJIs) Along the Length of a Nucleic Acid Molecule

Nucleotide primers used to execute the method of the invention, hereafter referred to as “spiky primers” (FIG. 3), are defined as nucleic acid constructs comprising DNA and/or RNA, each of which consists of three main features:
(1) Two anti-parallel, non-complementary oligonucleotide “feet” designed to anneal to the template DNA in tandem and hold the remainder of the construct on the template (i.e., template primers). The 3′-foot serves as a sequence-specific primer that initiates synthesis of the second strand in the pre-PCR duplication of the first strand.
(2) A double-stranded fully complementary region with random nucleotides that serve as a unique junction identifier (UJI), and its reverse complement. The pair consisting of a UJI and its reverse complement positioned on both sides of the PCR primer junction will uniquely label the sequences on the either side as belonging to the same junction, so that they can be recognized as such after the junction is partitioned during PCR. While most of a given spiky primer is made of synthetic DNA, the complementary UJI sequence (and any downstream sequences) in the spiky primer are made by elongation of the 3′-end of the “priming stem” by DNA polymerase (T4), which ensures that an exact copy of the UJI is being made. The UJIs are used to associate sub-fragments of the original template molecule.
(3) A region that incorporates universal primer sequences, which are used to amplify sub-fragments of the original template molecule.

### Example 4. Synthesis of Spiky Primers

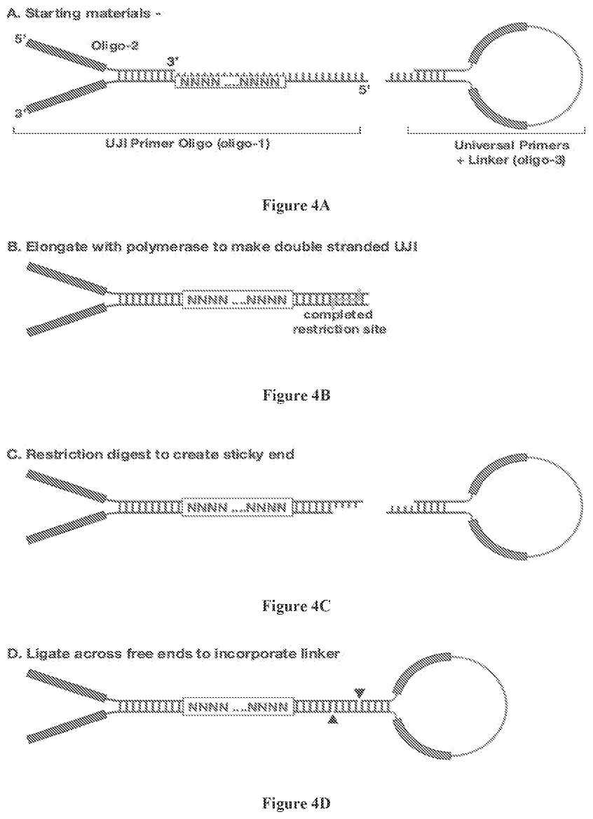

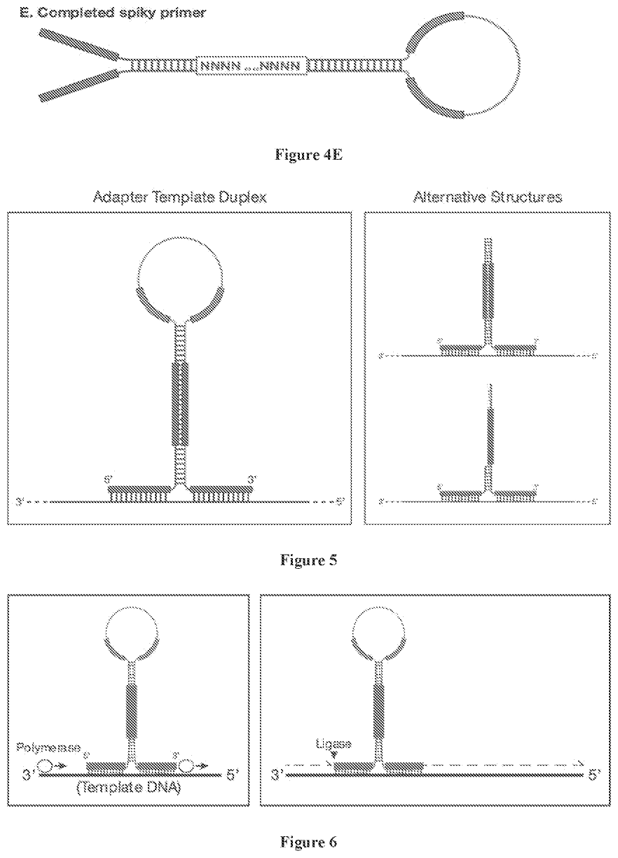

Each spiky primer requires three pre-synthesized components: 1) an oligonucleotide sequence, referred to here as oligo-1, with a UJI and 3′-foot (example 3); 2) an oligonucleotide sequence, referred to here as oligo-2, with a 5′-foot (example 3) and a sequence that is complementary to the sequence of oligo-1 between the UJI and the 3′-foot; and, 3) an oligonucleotide, referred to here as oligo-3, with a stem and a loop structure, that latter of which contains universal primer sequences (FIG. 4A). To synthesize the spiky primer, oligo-1 and oligo-2 are mixed and the complementary region on oligo-2 initiates formation of a double-stranded stem with oligo-1. Addition of polymerase extends the sequence from the 3′-end of oligo-2 to make a complete stem with a double-stranded UJI. The newly completed stem also contains a double-stranded restriction enzyme site (FIG. 4B). Application of the appropriate restriction enzyme creates a sticky end, which is complementary to the sticky end of oligo-3 (FIG. 4C). After annealing at the sticky end, addition of a ligase joins the single-strand nicks to complete the spiky primer (FIG. 4D). Completed primers (FIG. 4E) can be size-selected or otherwise filtered for purity to remove any unwanted ligation combinations or incomplete primers.

### Example 5. Method For Annealing Spiky Primers to the DNA Molecule of Interest

Spiky primers are first annealed to the template DNA either: (1) at periodic intervals determined by a priori knowledge of the template sequence; or, (2) by random oligonucleotide annealing (FIG. 5). In the case of a priori knowledge of the template sequence, the spiky primer feet are designed to have a melting temperature and no “off-target” sequences in the DNA mixture to ensure specificity of annealing of the primer only to the molecule of interest. A suitable high-fidelity polymerase without 5′-3′ strand displacement activity (e.g., T4, Q5) is used to elongate the DNA sequence from all free 3′-ends of DNA, filling in the gaps between the spiky primers along the length of the original molecule. In some embodiments, this step includes elongation of the priming stem discussed in Example 3 and Example 4 above.

### Example 6. Spiky Primer-Based PCR (Spiky-PCR) For Elongation and Ligation

When the polymerase elongating from the 3′-end of an upstream spiky primer meets the 5′-end of the next downstream spiky primer along the molecule, the lack of strand displacement activity causes the polymerase to stop and to fall off the template, leaving a nick between the nascent DNA chain and the 5′-end of the downstream spiky primer. The remaining nick is ligated using a high-fidelity DNA ligase (e.g., Hi Fi Taq ligase) (FIG. 6). Ligation of nicks 5′ to all spiky primers creates a continuous DNA strand covering the entire original template. Note that the 5′-most primer introduces only one UMI sequence, which serves as a terminal UMI. This UMI is distinct from the internal UJI paired sequences. Excess spiky primer sequences are removed using single-stranded DNA-specific 3′-5′/5′-3′ exonuclease VII (ExoVII).

Example 7. Removal of Incomplete Molecules and Non-Specific Products

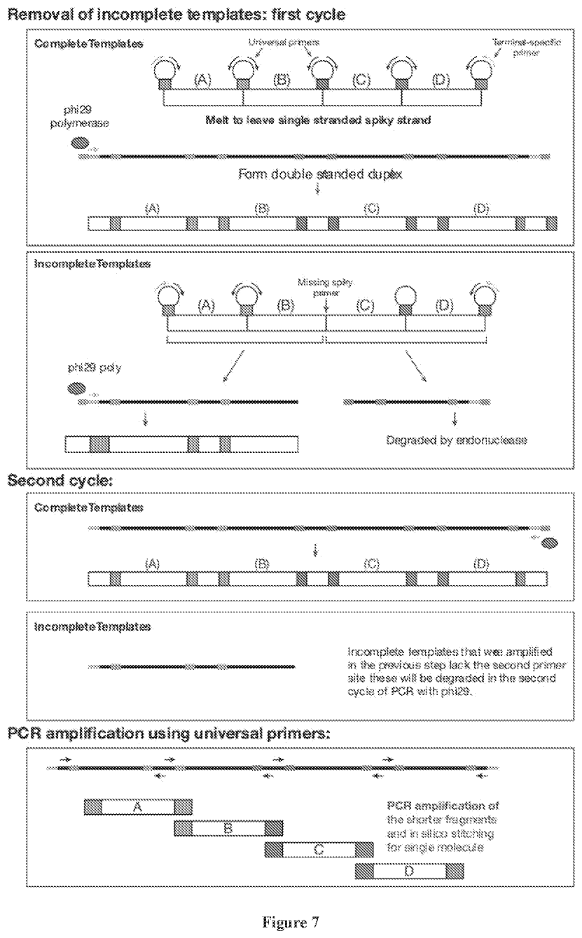

In cases of incomplete annealing, elongation and/or ligation at any of the steps involved in spiky-PCR (see Examples 3-6), incomplete DNA molecules and non-specific nucleic acid products would be generated. Where coverage of only the full-length original molecule is required for an experimental purpose, these incomplete templates should be eliminated before amplifying the spiky DNA (refer to Example 8 and Example 9 below). Otherwise, any incomplete fragment between two successfully incorporated spiky primers will be amplified. To remove this contamination (FIG. 7), spiky double-stranded DNA is denatured and a UMI-bearing primer complementary to the 3′-end of the nascent spiky strand is used to synthesize a full-length complementary strand with phi29 polymerase. Once the third strand is complete, any cDNAs with a 3+-end (including the complete cDNA) will be double stranded. In contrast, all other spiky fragments will remain single-stranded. These single-stranded molecules can be then eliminated by single stranded DNA-specific endonuclease treatment. This process (denaturing, UMI-bearing primer annealing, elongation with phi29 polymerase, and single-stranded endonuclease treatment) is repeated again for the 5′-end of the nascent template. In this way, the only duplexes that remain are complete molecules with fully incorporated spikes at all junctions. After the second round of endonuclease treatment, any incomplete molecules resulting from missing spiky primers or incomplete ligation will have been removed. This may be desired for subsequent downstream analyses of the spiky PCR-produced products.

For this protocol to work, nucleotide sequences of the terminal primer pairs (gray curved arrows in FIG. 7, upper two panels depicting Removal of incomplete templates: first cycle) need to be different from the universal primer pairs (black curved arrows in FIG. 7, upper two panels depicting Removal of incomplete templates: first cycle. The benefit of the above optional procedure is increased specificity and robustness of the entire procedure for certain applications. For example, in using the invention to assess viral genomes in clinical settings, viral nucleic acids may be mixed with host (e.g., human) DNA, which is very complex. In such a case, it is possible that spiky primers could anneal in the right orientation at short distances from each other on a non-target (host) genomic segment containing the universal primer sites; in turn, this could serve as contaminating/competing amplicons in the PCR reaction even if the initial presence of such a species is very low. All such nonspecific products formed, however, will lack the terminal primers needed to get second strand protection during phi29 replication, and thus these will be eliminated during the endonuclease step.

### Example 8. Generic PCR of Sub-Fragments

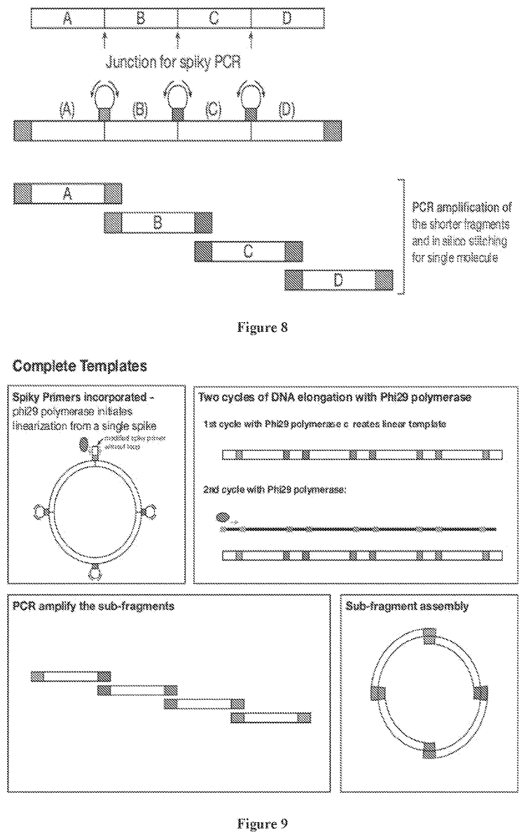

After incorporation of spiky primers with or without removal of incomplete templates, the template is subjected to PCR using universal primers to amplify each sub-fragment with universal primer sequences flanking the spikes. Because of the design of the spiky primer, adjacent fragments in the original template share the same random sequence (UJI), which therefore uniquely labels a given junction. The spiky primers also incorporate a universal PCR primer region, which allows for the amplification of UJI-flanked fragments (FIG. 8). After amplification, molecules are sequenced by any suitable sequencing platform. A computational algorithm is used to deconvolute the reads into consensus sequences, and the original template is determined by connecting consensus fragments sharing identical UJIs to generate a complete consensus sequence. In analysis of certain sequences, different sub-fragments of DNA may have different PCR efficiencies, resulting in uneven replication in a common PCR mixture. This caveat can be ameliorated by optimizing the fragments and conditions to achieve more balanced representation of all sub-fragments. For example, the PCR conditions can be adjusted to make it more difficult to amplify shorter-length sub-fragments shorter in length, which will compensate for the inherently lower amplification efficiency of longer sub-fragments compared to shorter sub-fragments in a common PCR mixture.

### Example 9. Application of Methods of the Invention For Analysis of Circular Nucleic Acid Molecules

With slight modifications, spiky PCR can be easily adapted to studying large circular DNA templates, including, but not limited to, bacterial genomes, mitochondrial DNA, chloroplast DNA, and plasmid DNA (FIG. 9). The primary change is that one of the spiky primers lacks the loop. Instead, this modified primer has either a terminal double-stranded stem or is “Y”-shaped, such that it has non-complementary ends. The modified spiky primer can be synthesized as detailed for the standard looped spiky primer (see Example 3 and Example 4), but with a modification to oligo-3 described in Example 4 to omit the linker region. The importance of this modification is that in the step for removing incomplete templates and non-specific products described in Example 7, the phi29 polymerase will complete the double-strand duplex and fall off. The resulting double-stranded linear sequence behaves as previously described. It is important to note that without this modification (viz. if all primers are normal spiky primers with loops), the phi29 polymerase will displace the 5′-end after it completes a full pass of the template. After endonuclease treatment, the displaced section will be degraded. Furthermore, there will be a termination point that is likely to disable one of the sub-fragments. The indicated modification to one of the primers ensures that the polymerase terminates after making one pass and does not displace the 5′-end. It also further reduces the likelihood of off-target or non-specific annealing because this modified primer would have to create a fully circular template. Another limiting factor in duplicating circular DNA is that torsion from the DNA helix structure is difficult to relieve. This is particularly relevant when introducing spiky primers, but also when releasing the spiky strand from the original circular template to allow the phi29 polymerase to work. This is not an impediment for practice of the invention, since single-stranded DNA-specific nickases can be used to target the non-template strand and relieve this torsion. Importantly, using a priori knowledge of the target DNA sequence, the nickases should be chosen to specifically degrade the circular sequence when it is not being used.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of generating a DNA/RNA duplex from a target RNA molecule, comprising incubating a plurality of reverse transcriptase primers (RT primers) and the target RNA molecule under conditions such that the target RNA molecule is reverse transcribed generating a DNA/RNA duplex,
wherein the plurality of RT primers are complementary to multiple annealing sites of the target RNA molecule such that each RT primer has an annealing site that is different than the annealing site of another RT primer in the plurality.

2. The method of claim 1, wherein the sequence of the target RNA molecule between two adjacent annealing sites is 1,000 to 7,000 nucleotides long.

3. The method of claim 2, wherein the sequence of the target RNA molecule between two adjacent annealing sites is about 1,000, 1,500, 2,000, 2,500, 3,000, 3,500, 4,000, 4,500, 5,000, 5,500, 6,000, 6,500, or 7,000 nucleotides long.

4. The method of claim 1, further comprising incubating an additional RT primer, wherein the additional RT primer comprises in 5′ to 3′ order:
(a) a first generic primer region having a nucleotide sequence that is not complementary to a sequence of the target RNA,
(b) a first unique molecular identifier (UMI-A) region, and
(c) a RT primer region that is complementary to the sequence located at the 3′ end region of the target RNA.

5. The method of claim 1, wherein the target RNA molecule is reverse transcribed via a reverse transcriptase.

6. The method of claim 5, wherein the reverse transcriptase is a processive reverse transcriptase.

7. The method of claim 5, wherein the reverse transcriptase reverse transcribes the sequence of the target RNA molecule between two adjacent annealing sites thereby generating complementary DNA fragments annealed to the target RNA molecule.

8. The method of claim 7, wherein the reverse transcriptase further reverse transcribes the adjacent annealing site thereby replacing the 5′ end of the adjacent fragment and creating excess single-stranded DNA.

9. The method of claim 8, further comprising trimming the excess single-stranded DNA via single-stranded DNA-specific exonuclease.

10. The method of claim 9, wherein the single-stranded DNA-specific exonuclease is single-stranded DNA-specific 3′-5′/5′-3′ exonuclease VII (ExoVII).

11. The method of claim 9, further comprising ligating the DNA fragments via ligase.

12. A method of generating a double-stranded cDNA molecule comprising the steps of:
(a) generating a DNA/RNA duplex according to the method of claim 1;
(b) treating the DNA/RNA duplex with RNase thereby removing the RNA; and
(c) incubating an adapter primer comprising a region that is complementary to the sequence located at the 3′ end region of the DNA under conditions such that a complementary DNA strand is formed thereby generating a double-stranded cDNA molecule.

13. The method of claim 12, wherein the RNase is RNase-H.

14. The method of claim 12, wherein the adapter primer, further comprises on the 5′ end in 5′ to 3′ order:
(a) a region complementary to a second generic primer having a nucleotide sequence that is not complementary to a sequence of the cDNA, and
(b) a region complementary to a second unique molecular identifier (UMI-B).

15. The method of claim 12, wherein the complementary DNA strand is formed via a DNA polymerase.

16-31. (canceled)

32. A method of detecting and removing an artificially recombined DNA molecule (chimera) resulting from PCR-jumping comprising:
(a) generating a double-stranded cDNA molecule according to the method of claim 12;
(b) amplifying the double-stranded cDNA molecule via a polymerase chain reaction using a first primer and a second primer that are complementary to the first generic primer region and the second generic primer region, respectively;
(c) sequencing the amplified double-stranded cDNA molecule;
(d) detecting the artificially recombined DNA molecule which does not have both UMI-A and UMI-B on the same double-stranded cDNA molecule; and
(e) removing the artificially recombined DNA molecule in silico.

33. A nucleic acid primer for sequencing a region of a target nucleic acid molecule comprising, in 5′ to 3′ order:
(a) a first specific primer region having a nucleotide sequence that is complementary to a first annealing site of the target nucleic acid molecule;
(b) a first unique junction identifier comprising random nucleotides;
(c) a first universal primer region having a nucleotide sequence that is not complementary to a sequence of the target nucleic acid molecule;
(d) a second universal primer region having a nucleotide sequence that is not complementary to a sequence of the target nucleic acid molecule;
(e) a second unique junction identifier comprising a nucleic acid sequence complementary to the first unique junction identifier; and
(f) a second specific primer region having a nucleotide sequence that is complementary to a second annealing site of the target nucleic acid molecule, wherein the second annealing site is adjacent to the first annealing site.

34-39. (canceled)

40. A nucleic acid primer for sequencing a region of a target nucleic acid molecule comprising, in 5′ to 3′ order:
(a) a first specific primer region having a nucleotide sequence that is complementary to a first annealing site of the target nucleic acid molecule;
(b) a first unique junction identifier comprising random nucleotides; and
(c) a second specific primer region having a nucleotide sequence that is complementary to a second annealing site of the target nucleic acid molecule, wherein the second annealing site is adjacent to the first annealing site.

41-47. (canceled)

48. A method of generating a nucleic acid product comprising incubating the nucleic acid primer claim 33 and a target nucleic acid molecule under conditions such that the nucleic acid product is formed.

49-72. (canceled)

73. A method of identifying the sequence of a target nucleic acid comprising:
(a) generating a nucleic acid product according to the method of claim 48;
(b) incubating a first specific primer and a second specific primer that are complementary to the first specific primer region and the second specific primer region of the nucleic acid primer and the nucleic acid product under conditions such that the nucleic acid product is amplified, thereby generating nucleic acid fragments that are flanked with unique junction identifiers;
(c) sequencing the nucleic acid fragments;
(d) assembling the nucleic acid fragments in silico, thereby identifying the sequence of the target nucleic acid.
